(19) United States
(12) Patent Application Publication
(10) Pub. No.: US 2013/0289339 A1
(43) Pub. Date: Oct. 31, 2013

(54) MEDICAL DEVICE AND MEDICAL DEVICE ASSEMBLY

(71) Applicant: TERUMO KABUSHIKI KAISHA, Ashigarakami-gun (JP)

(72) Inventor: Wataru Karino, Ashigarakami-gun (JP)

(73) Assignee: TERUMO KABUSHIKI KAISHA, Ashigarakami-gun (JP)

(21) Appl. No.: 13/871,610

(22) Filed: Apr. 26, 2013

(30) Foreign Application Priority Data
Apr. 27, 2012 (JP) 2012-103818

(51) Int. Cl. A61B 17/12; A61F 2/00

(52) U.S. Cl. CPC A61B 17/12099; A61F 2/0063
USPC 600/37

(57) ABSTRACT

A medical device includes a cover member and a holding member. The cover member includes: an accommodating section so provided as to be able to accommodate either one of a right lung and a left lung of a living body and freely deformable according to inflation and deflation of a lung which is accommodated in the state of accommodating the lung therein; and an opening through which the lung is introduced into the accommodating section. The holding member is provided at the opening of the cover member, deforms the aperture shape of the opening by having its oppositely located arbitrary portions brought closer to or away from each other, and so functions that the aperture area of the opening is thereby held constant before and after the deformation.

### CROSS-REFERENCE TO RELATED APPLICATION

This application is based on Japanese Patent Application No. 2012-103818 filed on Apr. 27, 2012, the contents of which are incorporated herein by reference.

### BACKGROUND

1. Technical Field

The present disclosure relates to a medical device and a medical device assembly for use in treatment of emphysema.

2. Description of Related Arts

Chronic obstructive pulmonary disease (COPD) means a diverse group of pulmonary diseases in which normal respiration is hampered, in other words, those diseases in which the lung or lungs are obstructed by the presence of at least one disease selected from among emphysema and chronic bronchitis. In COPD, these symptoms are often present simultaneously.

Of these diseases, the emphysema refers to the state in which the tissues called lung parenchyma inclusive of respiratory bronchioles, alveolar ducts, alveoli, and alveolar sacs are suffering abnormal expansion attended by breakage. While normal lung parenchyma shrinks (is deflated) at the time of expiration, emphysematous lung parenchyma would not return to its original state once expanded (inflated) by respiration. Therefore, the emphysematous lung parenchyma cannot perform sufficient exhalation. Moreover, the alveolar effective area and vascular bed (capillary vessels extending in all directions throughout the surfaces of alveoli) are decreased, so that ventilation capability of the lung as a whole is lowered. In addition, since elastin and the like are broken by inflammation, the lung is lowered in elasticity as well, to become unable to keep pulling and spreading the airway, so that the bronchus comes to be in an easily deformable state. Accordingly, when the lung shrinks at the time of exhalation, the bronchus is narrowed by being compressed by the surrounding alveoli filled with air, resulting in hyperinflation of the lung and difficult discharge of air.

As treatment of emphysema, at present, there are adopted the oxygen therapy and pharmacotherapy, which are methods for temporarily alleviating the symptom. In addition to these therapeutic methods, lung volume reduction surgery (LVRS) in which the lesion part of the lung is removed and expansion of the normal part of the lung is promoted is performed as a surgical method. In the lung volume reduction surgery, however, not only the lesion part but also much normal part in the vicinity of the lesion part may be removed. In addition, this surgery is highly invasive, and urges the patient to stay in the hospital for a prolonged time. Thus, the LVRS imposes a considerable burden on the patient.

U.S. Pat. No. 6,416,554 proposes a therapeutic method in which a hyperinflation state of a lung is mitigated by partly compressing part of the lung, without removing the lesion part of the lung. In this treating method, a cover member (jacket) which is preliminarily shaped so as to compress the lesion part of the lung is used. According to this method, a reduction in the lung volume can surely be achieved by compression of the lesion part. When the lesion part is compressed, however, an influence of the compression is imposed on the normal part in the vicinity of the lesion part. Consequently, this therapeutic method also causes a considerable burden on the patient.

On the other hand, International Publication (PCT) WO 98/01084 proposes a therapeutic method based on the use of a flexible cover member which is disposed so as to cover the lung as a whole. According to such a treating method, it is possible to reduce the lung volume, by the elasticity of the cover member, and to impart shrinkability to the lung. Besides, the influence on the normal part, as in the method disclosed in U.S. Pat. No. 6,416,554, is slight in this cover member method; consequently, a minimally invasive technique can be realized.

### SUMMARY

In the technique based on the use of the cover member disclosed in International Publication (PCT) WO 98/01084, the operation of putting the cover member on the lung is carried out through an opening formed in the cover member. Since the cover member is formed from a deformable material, twisting (kinking) or bending is liable to occur at the opening formed in the cover member. This makes it necessary for the operator to carry out the cover member putting operation with sufficient care such as to prevent the opening of the cover member from interfering with and being caught on the lung due to narrowing of the opening by twisting (kinking) or bending of the opening. In addition, if the opening is broadened excessively during the procedure, the cover member may interfere with and be caught on a biorgan present in the surroundings of the lung. In using the conventional cover member, therefore, it is necessary for the operator to put the cover member onto the lung while maintaining an appropriate aperture area of the opening in the inside of the living body. Thus, the operator is forced to achieve a very intricate technique. Besides, in order to achieve such a technique, it is difficult to keep small the incised part.

In order to reduce the intricateness of the technique or procedure as just-mentioned, it may well be contemplated, for example, that it suffices to preliminarily design the aperture area of the opening of the cover member to be comparable to the size of the lung, and to dispose at the opening a member by which the same aperture shape can be maintained before and after the putting of the cover member onto the lung. When such a configuration is adopted, however, it is also necessary to widely incise the living body at the time of introducing the cover member. Consequently, it is very difficult to realize a minimally invasive technique.

Thus, there is a need for a medical device and a medical device assembly by which it is made possible, in therapy of emphysema based on the use of a deformable cover member, to perform an operation of putting the cover member onto the lung easily and speedily and to realize a minimally invasive treatment of emphysema.

According to an embodiment of the present disclosure, there is provided a medical device including: a cover member including an accommodating section provided so as to be able to accommodate either one of a right lung and a left lung of a living body, the accommodating section being freely deformable according to inflation and deflation of a lung which is accommodated in the state of accommodating the lung therein, and an opening through which the lung is introduced into the accommodating section; and a holding member which is provided at the opening of the cover member, which deforms aperture shape of the opening by having its oppositely located arbitrary portions brought closer to or away from each other, and by which aperture area of the opening can be held constant before and after the deformation.

In this medical device, the aperture shape of the opening of the cover member can be changed by pushing or pulling opposed arbitrary portions of the holding member provided at the opening. In addition, it is possible, by the holding member, to prevent the aperture area of the opening from being narrowed or broadened in an unintended manner. This ensures that during the operation of putting the cover member onto the lung, the opening can be prevented from interfering with and being caught on the lung or a biorgan present in the surroundings of the lung. Besides, the aperture shape of the opening can be changed comparatively freely at the time of introducing the cover member into the living body. This makes it possible to reduce the size of the wound hole that is formed by incising the living body at the time of the introduction. Then, it is possible, by the cover member put on the lung, to reduce the lung volume and impart shrinkability to the lung. Therefore, the hyperinflation state of the emphysematous lung can be mitigated, and ventilation efficiency of the lung can be enhanced. Accordingly, in the treatment of emphysema based on the use of a deformable cover member, the operation of placing the cover member on the lung can be performed easily and speedily, and a minimally invasive treatment of emphysema can be realized.

In the medical device as above, preferably, a configuration is adopted wherein the holding member includes a main body section disposed along the opening, a connecting section provided on one end side of the main body section and being connectable with the main body section, and an extension section extending from a connection position between the main body section and the connecting section toward the other end side of the holding member; and the aperture area of the opening can be changed by changing the connection position.

According to this configuration, the aperture area of the opening can be changed by an operation of changing the connection position between the main body section and the connecting section of the holding member. Therefore, the aperture area of the opening can be easily controlled even after the introduction of the medical device into the living body. Thus, the technique based on the use of the medical device can be carried out more smoothly.

In the medical device as above, preferably, a configuration is adopted wherein the connecting section is so provided as to be movable along the main body section in the state of being connected to the main body section, and the aperture area of the opening can be changed by moving the connecting section.

This configuration ensures that the aperture area of the opening can be controlled by moving the connecting section of the holding member along the main body section. Therefore, an operation of controlling the aperture area can be conducted more smoothly.

In the medical device as above, preferably, the holding member has a lock member which permits the connection position to be so changed as to reduce the aperture area of the opening and prevents the connection position from being so changed as to enlarge the aperture area of the opening.

According to this configuration, it is possible to prevent the aperture area of the opening from being inadvertently enlarged by the lock member after the introduction of the cover member into the living body. Consequently, the cover member can be favorably prevented from falling off the lung, and the cover member can be kept indwelling in the living body stably for a long period of time.

In the medical device as above, preferably, the extension section is so configured as to be separable from the main body section.

This configuration enables the extension section of the holding member to be separated from the main body section. Therefore, after the introduction of the medical device into the living body, the extension section having become unnecessary can be separated from the main body section and taken out of the living body. Consequently, the medical device can be left indwelling in the living body in a compact form.

In the medical device as above, preferably, the cover member includes a net which is flexible.

According to this configuration, the cover member includes the flexible net. With the cover member put on the lung, therefore, the lung volume can be reduced by the shrinking force of the net. Further, shrinkability can be imparted to the lung. Therefore, a suitable treatment of emphysema can be realized.

In the medical device as above, preferably, the medical device is a medical device for treatment of emphysema, the medical device adapted to reduce the volume of an emphysematous lung and to impart shrinkability to the lung.

According to this configuration, it is possible to provide a medical device suitable for treatment of emphysema, which device is adapted to reduce the volume of an emphysematous lung and to impart shrinkability to the lung.

According to another embodiment of the present disclosure, there is provided a medical device assembly including: a medical device including a cover member including an accommodating section provided so as to be able to accommodate either one of a right lung and a left lung of a living body, the accommodating section being freely deformable according to inflation and deflation of a lung which is accommodated in the state of accommodating the lung therein, and an opening through which the lung is introduced into the accommodating section, and a holding member which is provided at the opening of the cover member, which deforms aperture shape of the opening by having its oppositely located arbitrary portions brought closer to or away from each other, and by which aperture area of the opening can be held constant before and after the deformation; a first introduction member including a flexible elongated member, a gripping section provided on a distal side of the elongated member and capable of gripping the holding member of the medical device, and a hand operation section provided on a proximal side of the elongated member and operable to control a gripping action of the gripping section; and a second introduction member including a tubular elongated member formed therein with an insertion lumen in which the extension section of the holding member can be inserted and passed.

This medical device assembly includes the medical device, the first introduction member, and the second introduction member. This ensures that in a technique for introducing the medical device into a living body and leaving the medical device indwelling in the living body, the first introduction member and the second introduction member can be used. Accordingly, the technique can be carried out more easily, speedily and less invasively.

In the medical device assembly as above, preferably, the second introduction member further includes a cutter having a cutting edge part capable of cutting the holding member of the medical device.

According to this configuration, an arbitrary portion of the holding member can be cut by the cutter possessed by the second introduction member, after the introduction of the medical device into the living body. Therefore, it is possible to perform such operations as control of the length of the holding member or cutting away an unnecessary part, after the introduction. Consequently, convenience of the technique based on the use of the medical device can be enhanced.

In the medical device assembly as above, preferably, at least one of the holding member of the medical device, the first introduction member, and the second introduction member is provided with a radiopaque part.

According to this configuration, at least one of the holding member of the medical device, the first introduction member, and the second introduction member is provided with the radiopaque part. This ensures that the introduction of each of the component members into the living body can be carried out more smoothly under radioscopy.

According to an embodiment of the present disclosure, there is provided a method of treating emphysema to reduce a lung volume and to impart shrinkability to a lung by using a cover member which includes an accommodating section being freely deformable according to inflation and deflation of the lung in the state of accommodating the lung therein, wherein the lung is introduced into the accommodating section through an opening which the cover member includes with the aperture area of the opening being held constant and the cover member is kept indwelling in the living body with the lung being covered by the accommodating section.

In this method, the aperture shape of the opening of the cover member can be changed by pushing or pulling opposed arbitrary portions of the holding member provided at the opening. In addition, it is possible, by the holding member, to prevent the aperture area of the opening from being narrowed or broadened in an unintended manner. This ensures that during the operation of putting the cover member onto the lung, the opening can be prevented from interfering with and being caught on the lung or a biorgan present in the surroundings of the lung. Besides, the aperture shape of the opening can be changed comparatively freely at the time of introducing the cover member into the living body. This makes it possible to reduce the size of the wound hole that is formed by incising the living body at the time of the introduction. Then, it is possible, by the cover member put on the lung, to reduce the lung volume and impart shrinkability to the lung. Therefore, the hyperinflation state of the emphysematous lung can be mitigated, and ventilation efficiency of the lung can be enhanced. Accordingly, in the treatment of emphysema based on the use of a deformable cover member, the operation of placing the cover member on the lung can be performed easily and speedily, and a minimally invasive treatment of emphysema can be realized.

### BRIEF DESCRIPTION OF THE DRAWINGS

FIG. 1 is a perspective view showing the general configuration of a medical device according to an embodiment of the present disclosure;

FIGS. 2A and 2B illustrate a holding member possessed by the medical device, wherein FIG. 2A is a plan view of the holding member, and FIG. 2B is a side view of the holding member;

FIGS. 3A to 3C illustrate operation of the holding member possessed by the medical device, wherein FIG. 3A shows a state before deformation of aperture shape of an opening, FIG. 3B shows a state after deformation of the aperture shape, and FIG. 3C shows a state after the aperture area of the opening is controlled;

FIGS. 4A and 4B illustrate a first introduction member to be used together with the medical device, wherein FIG. 4A is a plan view showing the general configuration of the first introduction member, and FIG. 4B is a partial enlarged sectional view showing a gripping section of the first introduction member;

FIGS. 5A and 5B illustrate a second introduction member to be used together with the medical device, wherein FIG. 5A is a partly sectional view showing the general configuration of the second introduction member, and FIG. 5B is a partially sectional view showing a state in which an extension section of the holding member is cut by a cutter possessed by the second introduction member;

FIGS. 6A and 6B are partially sectional views showing a modification of the cutter possessed by the second introduction member;

FIG. 7 illustrates in a simplified form a living body and lungs in the living body to which the medical device is to be applied;

FIG. 8 illustrates operations of the medical device and a medical device assembly, showing in an enlarged form a lung in a state before the medical device is mounted thereto;

FIG. 9 illustrates operations of the medical device and the medical device assembly, showing a state in which the medical device has been introduced into the vicinity of the lung;

FIG. 10 illustrates operations of the medical device and the medical device assembly, showing an operation of introducing the lung into an accommodating section of the medical device;

FIG. 11 illustrates operations of the medical device and the medical device assembly, showing an operation of introducing the lung into the accommodating section of the medical device;

FIG. 12 illustrates operations of the medical device and the medical device assembly, showing an operation of controlling the aperture area of the opening after the introduction of the lung into the accommodating section of the medical device;

FIG. 13 illustrates operations of the medical device and the medical device assembly, showing an operation of reducing the aperture area of the opening by pulling an extension section of the holding member in the outside of the living body;

FIG. 14 illustrates operations of the medical device and the medical device assembly, showing in a simplified form the living body in a state in which the medical device is left indwelling in the living body; and

FIG. 15 is a partly sectional view for illustrating a medical device introducing procedure in introducing the medical device into the living body by the first instruction member and the second introduction member.

### DETAILED DESCRIPTION OF THE PREFERRED EMBODIMENTS

Now, embodiments of the present disclosure will be described below, referring to the drawings. Incidentally, dimensional ratios in the drawings are exaggerated for convenience of explanation, so that the dimensional ratios may be different from the actual ratios.

First, the configurations of components of a medical device and a medical device assembly according to the present embodiment will be described.

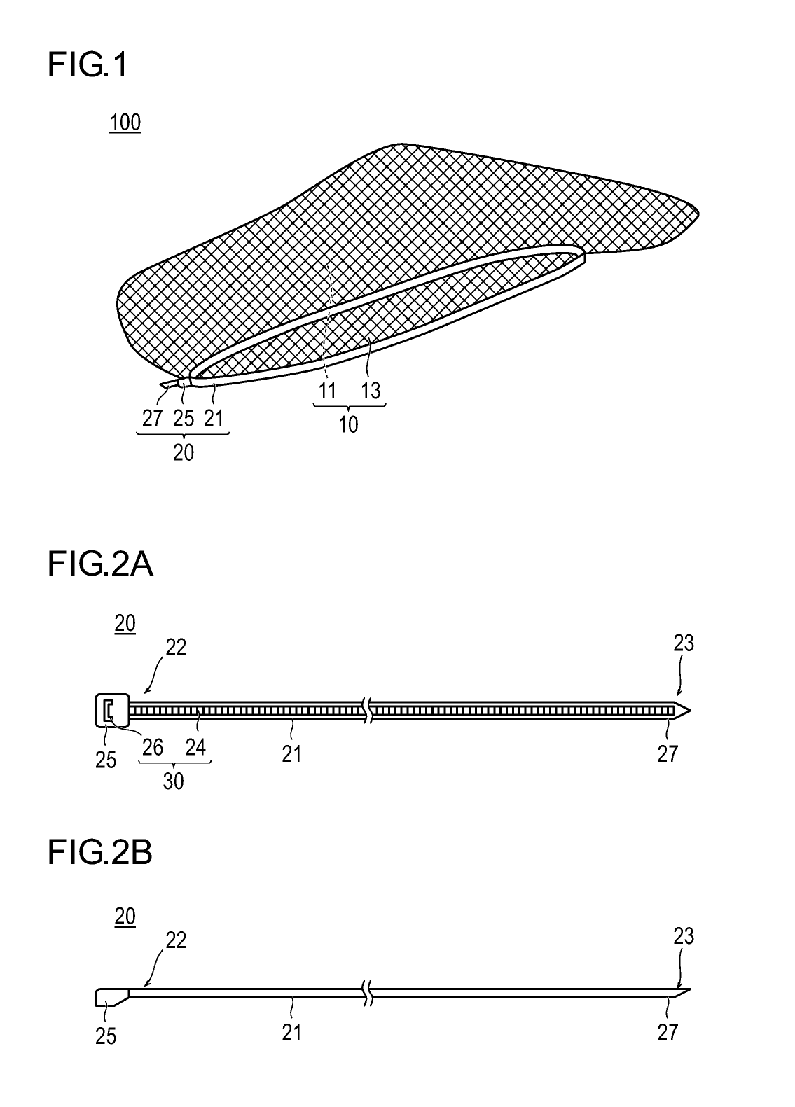

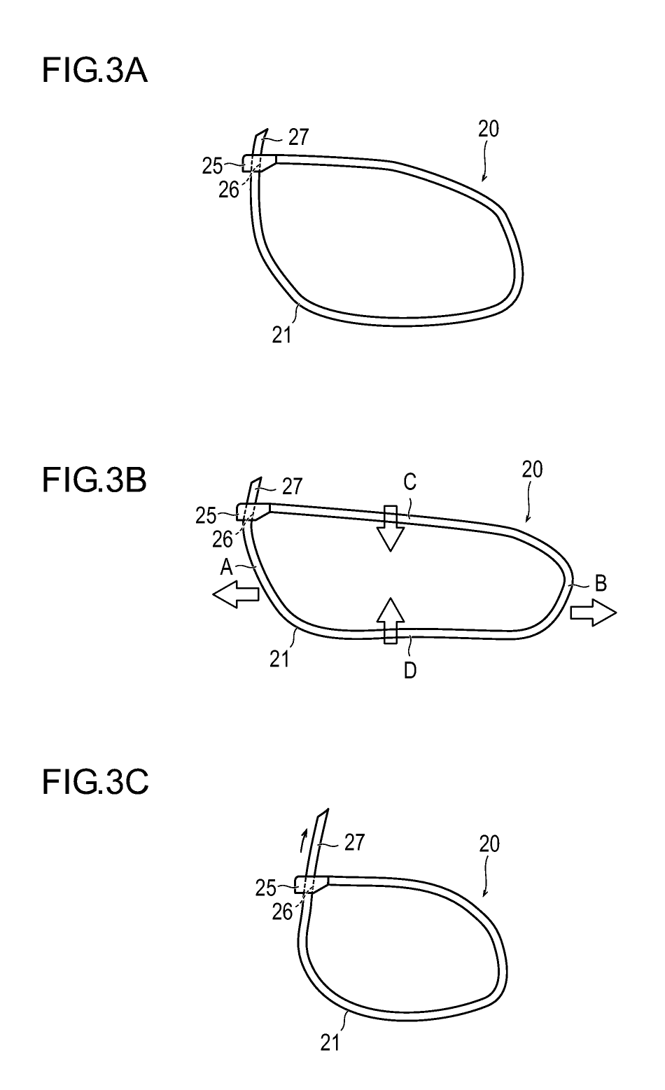

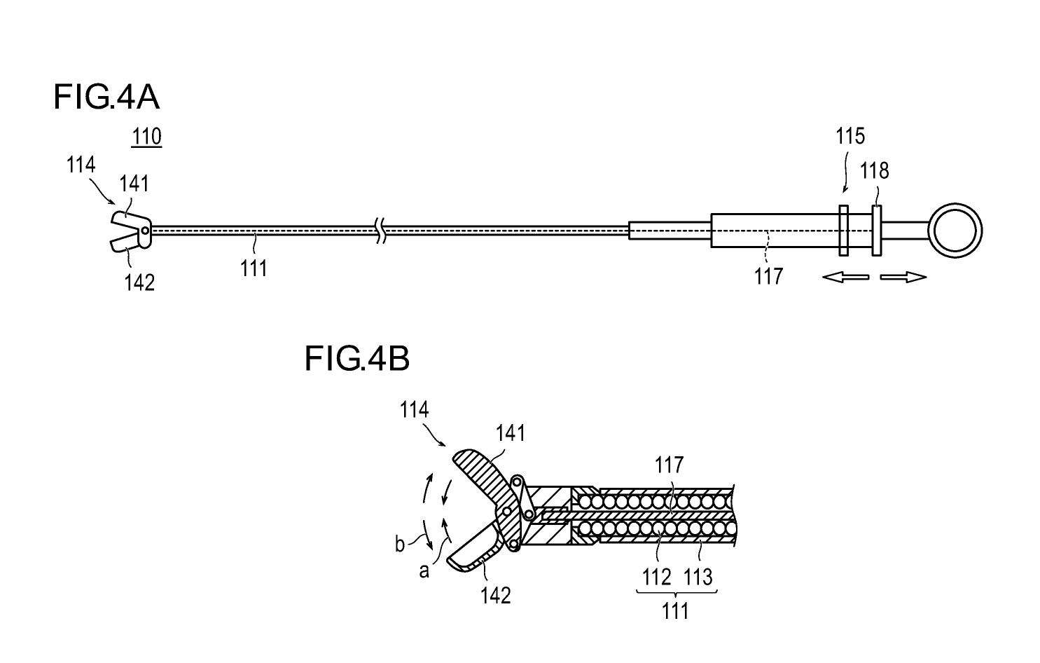

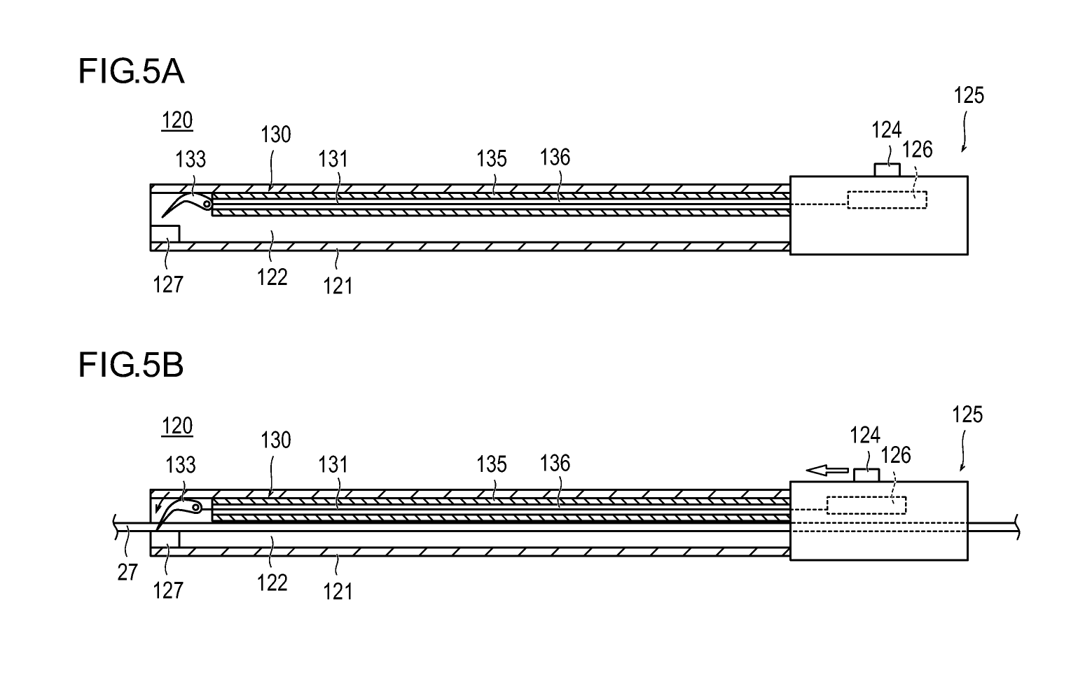

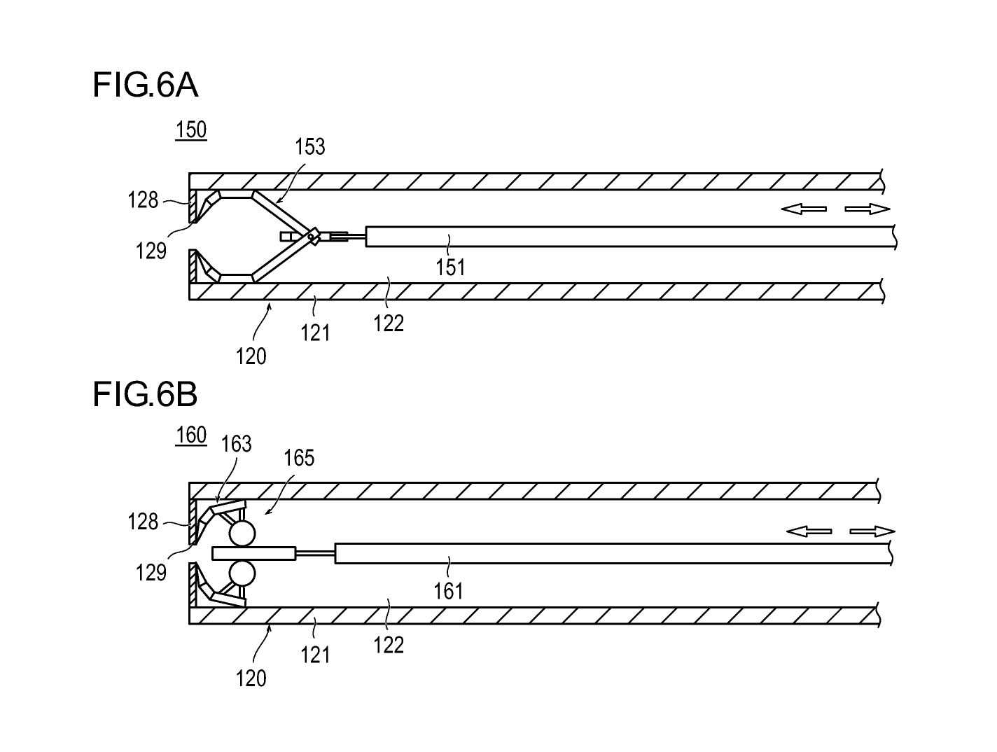

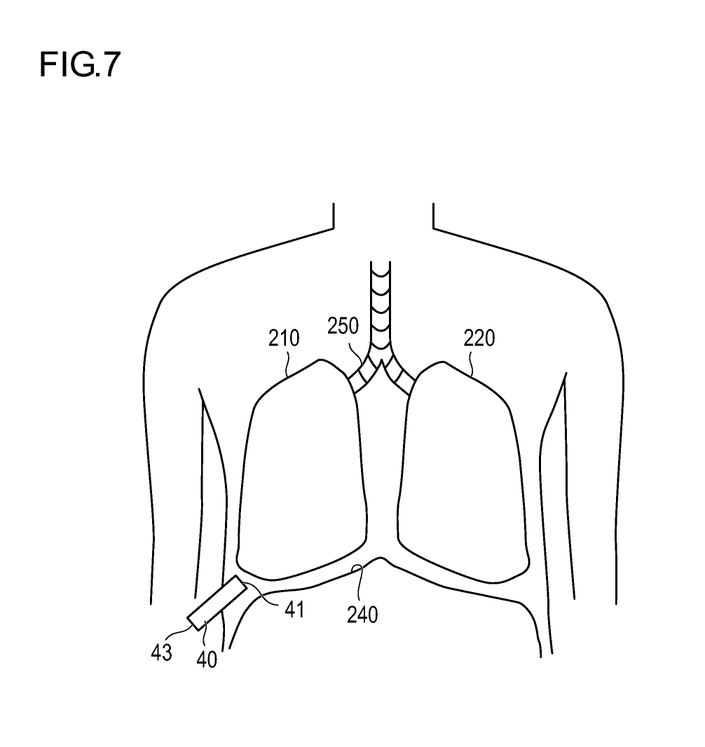

FIG. 1 is a view showing the general configuration of the medical device; FIGS. 2A to 3C are views for illustrating a holding member possessed by the medical device; FIGS. 4A and 4B are views for illustrating a first introduction member constituting the medical device assembly together with the medical device; FIGS. 5A and 5B are views for illustrating a second introduction member constituting the medical device assembly together with the medical device; FIGS. 6A and 6B are views showing a modification of a cutter possessed by the second introduction member; and FIGS. 7 to 15 are views for illustrating operations of the medical device and the medical device assembly.

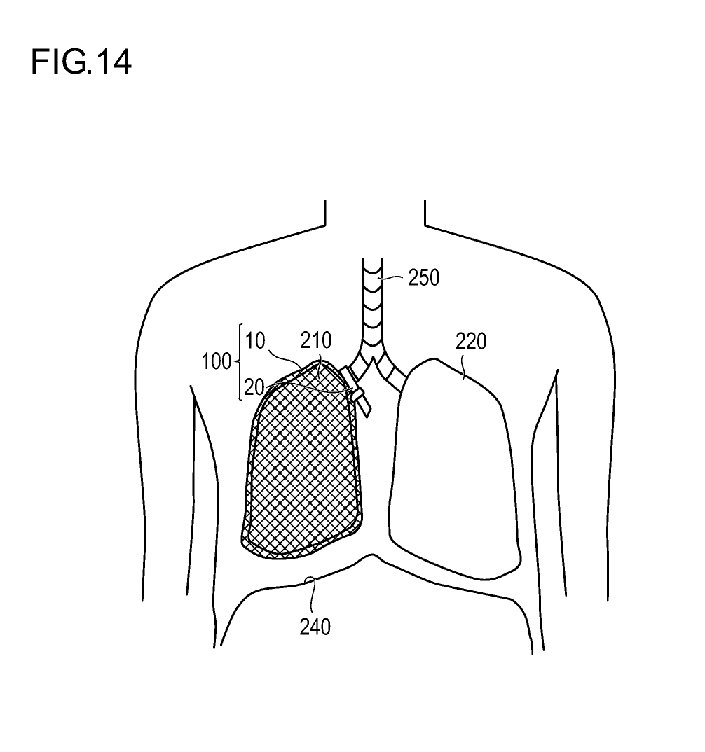

As shown in FIGS. 1 and 14, the medical device 100, when outlined, includes a cover member 10 and a holding member 20. The cover member 10 includes an accommodating section 11 so provided as to be able to accommodate either one of a right lung 210 and a left lung 220 of a living body, and an opening 13 through which to introduce the lung into the accommodating section 11. The holding section 20 is provided at the opening 13 of the cover member 10.

The medical device 100 is a medical device for treatment of emphysema, adapted to reduce the volume of an emphysematous lung accommodated in the accommodating section 11 of the cover member 10, and to impart shrinkability to the lung accommodated in the accommodating section 11. In this embodiment, a single lung composed of an upper lobe, a middle lobe and a lower lobe, or an upper lobe and a lower lobe, which are located in a thoracic cavity surrounded by a thoracic cage, will be referred to in its entirety as the right lung 210 or the left lung 220, correspondingly. In addition, while an application example in which the medical device 100 is applied to the right lung 210 will be described in this embodiment, as shown in FIG. 14, but the object to which the medical device 100 is applied may naturally be either of the right lung 210 and the left lung 220.

As shown in FIG. 1, the accommodating section 11 constitutes a space defined inside the cover member 10, and can be freely deformed according to the expansion (inflation) and contraction or shrinkage (deflation) of the lung in the condition where the lung is accommodated in the space. The volume of the accommodating section 11 is set to a size for reducing the volume of the lung by contact of the inner surface of the accommodating section 11 with the outer surface of the lung. For instance, where the medical device 100 is used for the right lung 210 or the left lung 220, the accommodating section 11 may be formed to have a volume of 1,000 to 4,000 cm³, preferably 1,500 to 3,000 cm³. It should be noted here, however, that the lung volume differs from patient to patient and according to the progress of emphysema, so that the volume of the accommodating section 11 can be arbitrarily set within such a range that the lung can be accommodated in the accommodating section 11 and the lung volume can be reduced thereby.

The cover member 10 may be composed, for example, of a net which is flexible or elastic. The material of the net is not specifically restricted so long as it is flexible or elastic. Examples of the material include polyethylene, polypropylene, nylon, polyester elastomers, silicone, synthetic rubbers, and various biodegradable materials. The interval and size of the meshes formed in the net are not particularly limited, and can be controlled as required. In addition, the internal shape of the accommodating section 11 of the cover member 10 can be set, for example, to be roughly the same as the external shape of the lung to which the cover member 10 is to be applied. With the internal shape of the accommodating section 11 set to such a shape, the cover member 10 can be disposed in fit to the whole part of the lung, whereby it is possible to effectively reduce the lung volume and to effectively impart shrinkability to the lung.

The opening 13 formed in the cover member 10 is composed of a hole having a predetermined shape. The opening 13 is configured to have such an aperture area that the lung can be passed through the opening 13. As will be described later, in the medical device 10, the aperture area of the opening 13 is so configured that it can be controlled.

As shown in FIGS. 1 to 3C, the holding member 20 can be composed of an elongated member which includes: a main body section 21 disposed along the opening 13; a connecting section 25 provided on one end side 22 of the main body section 21 and being connectable with the main body section 21; and an extension section 27 extending from the connection position between the main body section 21 and the connecting section 25 toward the other end side 23 of the holding member 20.

The material forming the holding member 20 is not specifically restricted. Examples of the material include such materials as nylon, fluoro-resins, PEEK, PVDF, polypropylene, and polyethylene. Preferably, the material of the holding member 20 is a material which can be cut by a cutter 130 possessed by a second introduction member 120 which will be described later. In addition, the whole part of the holding member 20 or part of the holding member 20 may be provided, for example, with radiopacity so that introduction of the medical device 100 into a living body can be performed radioscopically.

The connecting section 25 has a groove 26 provided on the one end side 22 of the holding member 20. Into the groove 26, an end portion on the other end side 23 of the holding member 20 can be inserted. With the end portion inserted in the groove 26, the whole part of the holding member 20 can be shaped into an annular shape (see FIG. 3A). In addition, the main body section 21 of the holding member 20 is provided with engaging parts 24 to be engaged with the groove 26. By the engagement of the engaging part 24 with the inner surface of the groove 26, movement of the main body section 21 inserted in the groove 26 is restricted. Therefore, when the main body section 21 is inserted in the groove 26, the main body section 21 is prevented from slipping out of the groove 26, and the shape of the holding member 20 is maintained in an annular shape. The end portion on the other end side 23 of the holding member 20 can be tapered off, as shown in the drawings, so as to ensure that it can be smoothly inserted into the groove 26.

The extension section 27 of the holding member 20 is composed of a predetermined portion on the other end side 23 of the main body section 21 that is led out of the groove 26 in the connecting section 25 (see FIG. 3A). In other words, as shown in FIGS. 2A and 2B, the extension section 27 constitutes part of the main body section 21 until the main body section 21 is inserted into the groove 26 in the connecting section 25. Incidentally, in the case where the holding member 20 is formed of a material which can be cut by the cutter 130 possessed by the second introduction section 120 to be shown in FIGS. 5A and 5B, the extension section 27 can be cut and separated from the main body section 21.

Operation of the holding member 20 will now be described, referring to FIGS. 3A to 3C.

FIG. 3A shows a state in which the main body section 21 and the connecting section 25 are connected with each other, whereby the shape of the holding member 20 is maintained in a predetermined shape. In the medical device 100, in the condition where the holding member 20 is thus shaped, the main body section 21 of the holding member 20 is attached to the cover member 10 along the opening 13 of the cover member 10 (see FIG. 1). The method for the attachment can be appropriately selected from among such methods as adhesion (with an adhesive) and fusion bonding, according to the materials of the holding member 20 and the cover member 10.

As shown in FIG. 3B, when arbitrary portions at point A and point B located opposite to each other, of the holding member 20, are pulled away from each other starting from the condition of FIG. 3A, the aperture shape of the opening 13 is changed (deformed). In this instance, the aperture area of the opening 13 is kept constant before and after the deformation. When the pulled state is released, the holding member 20 returns into the original shape shown in FIG. 3A, and the opening 13 of the cover member 10 also returns into the original shape accordingly. Incidentally, also in the case where arbitrary portions at point C and point D located opposite to each other, of the holding member 20, are pushed toward each other, it is possible to change (deform) the aperture shape of the opening 13 while keeping constant the aperture area, in the same manner as above. In the medical device 100, in order to ensure that change (deformation) of the aperture shape of the opening 13 can be operated by the holding member 20 and the aperture area of the opening 13 can be kept constant before and after the deformation by the holding member 20 as above-mentioned, the rigidity of the holding member 20 is preferably set higher than the rigidity of the cover member 10.

As shown in FIG. 3C, by changing the connection position between the main body section 21 and the extension section 27, it is possible to reduce the diameter of the loop of the holding member 20, and to reduce the aperture area of the opening 13 of the cover member 10 accordingly. Here, the connecting section 25 may be so configured as to be movable along the main body section 21 in the state of being connected with the main body section 21. In the holding member 20, the connecting section 25 can be slid along the main body section 21, by operating the connecting section 25 and the extension section 27 to move away from each other in the condition where the main body section 21 is inserted in the groove 26 possessed by the connecting section 25. By such an operation, the connection position can be changed, whereby the aperture area of the opening 13 of the cover member 10 can be controlled to decrease.

The holding member 20 may be provided with a lock member 30 which permits the connection position to be so changed as to reduce the aperture area of the opening 13 of the cover member 10 and which prevents the connection position from being so changed as to enlarge the aperture area of the opening 13. In the holding member 20, the lock mechanism 30 is composed of the groove 26 possessed by the connecting section 25 and the engaging parts 24 formed in the main body section 21 (see FIG. 2A). The engaging parts 24 are formed in rugged shape (projection-and-recess shape) so as to fit to the inner surface of the groove 26 when moved in an inserting direction within the groove 26. Accordingly, the connecting section 25 can be moved in the inserting direction but is restricted in movement in the direction opposite to the inserting direction. This ensures that once the main body section 21 is inserted into the groove 26, the connection position is prevented from being so changed as to enlarge the aperture area of the opening 13 of the cover member 10. Incidentally, in the holding member 20, the force with which the inner surface of the groove 26 and the engaging part 24 of the main body section 21 are engaged with each other can be appropriately controlled, in order to prevent the main body section 21 from moving inadvertently in the inserting direction in the condition where the main body section 21 is inserted in the groove 26. Besides, a deforming mechanism for deforming the inner surface of the groove 26 may be provided as a configuration for releasing the lock based on the engagement.

The aperture area of the opening 13 is not particularly limited insofar as the lung can be introduced into the accommodating section 11 via the opening 13. In the case where the medical device 100 is used for the right lung 210 and the left lung 220, the aperture area may be, for example, 100 to 800 cm², preferably 250 to 550 cm².

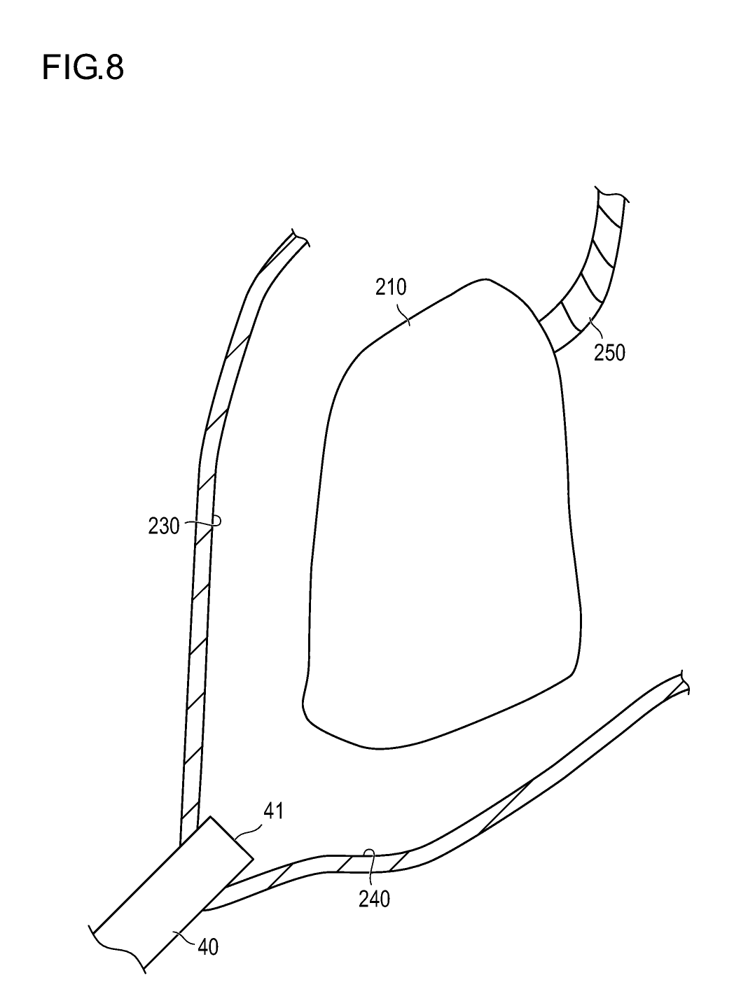

The aperture shape of the opening 13 is preferably set to be roughly elliptic in the state before the cover member 10 is put on the lung, in other words, in the state as shown in FIG. 3A before the aperture shape is changed (deformed). The reason is as follows. For example, in the case where an operation of putting the cover member 10 onto the right lung 210 is conducted by introducing the cover member 10 into the living body from a lateral side of the right lung 210 and moving the cover member 10 along the width direction of the right lung 210 (in the left-right direction in the drawings) as shown in FIGS. 8 to 11, the operation can be carried out more smoothly by conforming the major axis direction of the opening 13 formed in the roughly elliptic shape to the height direction of the right lung 210 (the vertical direction in the drawings). It should be noted here, however, that the aperture shape of the opening 13 can be deformed according to the deformation of the holding member 20 as above-mentioned, so that the aperture shape is not specifically restricted but may be set in an arbitrary shape.

Now, a first introduction member 110 and the second introduction member 120 which are used for introducing the medical device 100 into a living body will be described below. The first introduction member 110 and the second introduction member 120 constitute a medical device assembly for treatment of emphysema, together with the medical device 100.

As the first introduction member 110, there can be used, for example, a member of such a structure as biopsy forceps known in the medical field. The configuration of the first introduction member 110 will be outlined. As shown in FIGS. 4A and 4B, the first introduction member 110 includes: an elongated member 111 which is flexible; a gripping section 114 provided on the distal side of the elongated member 111 and capable of gripping the holding member 20 of the medical device 100; and a hand operation section 115 provided on the proximal side of the elongated member 111 and operable to control the gripping action of the gripping section 114. The whole part of the first introduction section 110 or parts of the first introduction section 110 may be provided with radiopacity so that the introduction of the medical device 100 into the living body can be performed radioscopically.

The gripping section 114 includes a first pinching member 141 and a second pinching member 142 which grip a predetermined member by coming closer to each other and which releases the grip by coming away from each other. The first pinching member 141 and the second pinching member 142 are connected with one end of an operating wire 117 for effecting a gripping action (indicated by arrows a in FIG. 4B) and a grip-releasing action (indicated by arrows b in FIG. 4B) of the gripping section 114. The other end of the operating wire 117 is connected to a sliding member 118 provided in the hand operation section 115. Various actions of the gripping member 114 can be actuated by pushing and pulling the sliding member 118. In an example of the action, when the sliding member 118 is slid proximally, a gripping action of the gripping section 114 can be actuated. In another example of the action, when the sliding member 118 is slid distally in a pushing-forward manner, a grip-releasing action of the gripping section 114 can be actuated.

The first pinching member 141 and the second pinching member 142 may be provided, at their mutually facing positions, with rugged projections for preventing a predetermined member in a gripped state from slipping off the position between the first pinching member 141 and the second pinching member 142. In addition, a lock mechanism for maintaining the relevant action of the gripping section 114 in the state of gripping a predetermined member or in the state of releasing the grip on a predetermined member may be added to the hand operation section 115 or the like.

The elongated member 111 possessed by the first introduction member 110 includes a main body section 112 composed of a coil, and a cover material 113 covering the main body section 112. The operating wire 117 may be disposed, for example, in the state of being inserted and passed in the inside of the main body section 112, as shown in the drawing.

Examples of the cover material 113 possessed by the elongated member 111 include polyvinyl chloride, polyvinyl acetate, polyethylene-vinyl acetate copolymer, polyethylene, polypropylene, cyclic polyolefins, polybutadiene, polyurethane, polyurethane elastomers, polyimides, polyamides, polyether block amide copolymer, polyesters, polyester elastomers, silicone resins, fluoro-resins such as polytetrafluoroethylene, etc., and polyether-ether ketone resins.

As shown in FIGS. 5A and 5B, the second introduction member 120 includes a tubular elongated member 121 formed therein with an insertion lumen 122 in which the extension section 27 of the holding member 120 provided on the cover member 10 can be inserted and passed. The second introduction member 120 may be provided with the cutter 130 having a cutting edge part 133 capable of cutting the extension section 27 of the holding member 20. Incidentally, the whole part of the second introduction member 120 or parts of the second introduction member 120 and the cutter 130 may be provided with radiopacity, in order to ensure that the introduction of the medical device 100 into the living body can be performed radioscopically, in the same manner as in the case of the first introduction member 110.

At the proximal end of the elongated member 121, a hand operation section 125 is provided which includes a lever 124 for controlling a cutting action on the extension section 27 by the cutter 130. The lever 124 is attached to a base member 126 which is so disposed that it can be advanced and retracted, distally and proximally, within the hand operation section 125. The base member 126 is connected to the cutting edge part 133 through an operating wire 131 for controlling the action of the cutter 130. Though not shown in the drawings, the hand operation section 125 is formed therein with a lumen in which the extension section 27 passed through the insertion lumen 122 of the elongated member 121 can be inserted and passed. This ensures that the extension section 27 can be led out from a proximal portion of the hand operation section 125 (see FIG. 5B).

The cutting edge part 133 possessed by the cutter 130 is formed in a curved outer shape, and can cut the extension section 27 of the holding member 20 by a distal portion (tip portion) thereof. The operating wire 131 connected to the cutting edge part 133 is passed through a lumen 136 of an inner tube 135 disposed in the insertion lumen 122 of the elongated member 121, and is connected to the base member 126 in the inside of the hand operation section 125.

The elongated member 121 is provided with a support member 127 for supporting the extension section 27 introduced into the insertion lumen 122. As shown in FIG. 5B, the extension section 27 is cut by the cutter 130 in the following manner. First, the extension section 27 is introduced into the insertion lumen 122 of the elongated member 121. Furthermore, a predetermined portion of the extension section 27 is led out from the proximal end of the hand operation section 125. Then, the extension section 27 is located on the support member 127, and, in this condition, the lever 124 is slid distally. Attendant on the sliding, the extension section 27 is cut by being nipped it between the cutting edge part 133 of the cutter 130 and the support member 127, with an upper surface portion of the cutting edge part 133 being guided by the inner wall of the elongated member 121. The extension section 27 thus cut can be recovered together with the second introduction member 120, in the state of being accommodated in the insertion lumen 122.

As materials forming the elongated member 121, the inner tube 135, the support member 127 and the like possessed by the second introduction member 120, there can be used, for example, the same or similar materials as those applicable as the cover material 113 of the first introduction member 110.

FIGS. 6A and 6B illustrate modifications of the cutter 130. It suffices for the cutter used in the embodiment to have at least a configuration such as to be able to cut the extension section 27 of the holding member 20. Thus, a configuration other than the configuration for cutting with a single cutting edge as above-mentioned can be adopted, as required. For instance, configurations structured to have a cutting edge for cutting the extension section 27 while pinching the extension section 27, as shown in FIGS. 6A and 6B, can be adopted.

A cutter 150 shown in FIG. 6A is provided at its distal end with a pair of cutting edge parts 153. When a shaft member 151 capable of being advanced and retracted along the axial direction is pushed distally, the cutting edge parts 153 are moved closer to each other, to cut the extension section 27. When the shaft member 151 is pulled proximally, the cutting edge parts 153 are moved away from each other, to perform an opening action. In addition, a cutter 160 provided with a rack-pinion mechanism 165 as shown in FIG. 6B (shown in simplified form) can also be adopted. The cutter 160 is provided at its distal end with a pair of cutting edge parts 163. When a shaft member 161 is pushed distally, the cutting edge parts 163 are moved away from each other, to perform an opening action. When the shaft member 161 is pulled proximally, the cutting edge parts 163 are moved closer to each other, to cut the extension section 27. Either of these cutters 150 and 160 is used together with the second introduction member 120, whereby a function of cutting the extension section 27 can be added to the second introduction member 120, like in the case of using the cutter 130 shown in FIGS. 5A and 5B.

In the case where either of the cutters 150 and 160 shown in FIGS. 6A and 6B is used in the state of being incorporated in the second introduction member 120, a guide section 129 formed with an opening 128 may be provided on the elongated member 121 possessed by the second introduction member 120. Where the guide section 129 is thus provided, the extension section 27 can be introduced into the insertion lumen 122 of the elongated member 121 easily and assuredly. In addition, where the position of the opening 128 is matched to the position between the cutting edge parts 153 possessed by the cutter 150 or the position between the cutting edge parts 163 possessed by the cutter 160, the extension section 27 passed in the opening 128 can be easily positioned between the cutting edge parts 153, 163, so that the operation of cutting the extension section 27 can be carried out more easily.

Now, referring to FIGS. 7 to 15, the procedure of a technique of using the medical device 100 and the medical device assembly according to this embodiment will be described below.

First, as shown in FIGS. 7 and 8, a port 40 through which the exterior of the living body and the vicinity of the right lung 210 of the living body communicate with each other percutaneously is provided, prior to the introduction of the medical device 100 into the living body. As the port 40, there can be used one that is composed of a tubular member having a distal opening 41 disposed inside the living body and a proximal opening 43 disposed outside the living body. At the proximal opening 43, there can be provided, for example, an air-tight type slit valve element which prevents inflow of air into the thoracic cavity while permitting only the insertion of the medical device 100, the first introduction member 110, and the second introduction member 120.

Of the port 40, a predetermined portion on the distal side is introduced into the living body through a hole formed by incision of the living body. Incidentally, as the material for the tubular member constituting the port 40, there can be used materials which are the same or similar to those for catheters and the like known in the medical field. Examples of the material include thermoplastic resins such as polyolefins such as polyethylene, polypropylene, ethylene-propylene copolymer, ethylene-vinyl acetate copolymer, etc., nylon, fluoro-resins, flexible polyvinyl chloride, etc., and various rubbers such as silicone rubber and latex rubber.

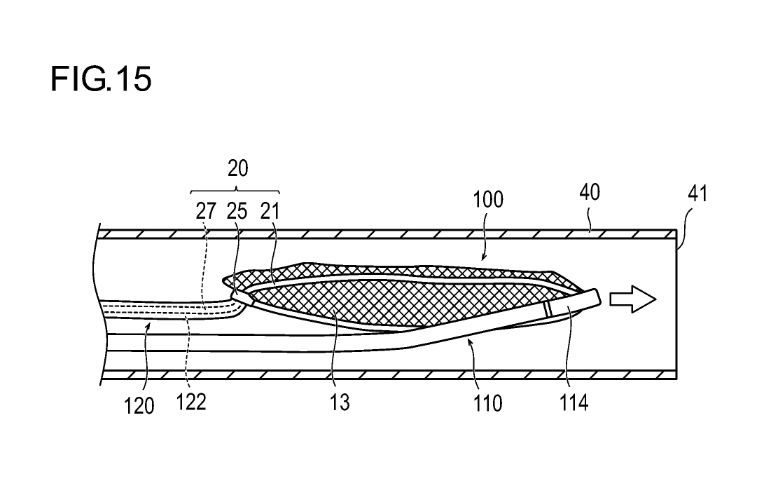

Next, as shown in FIG. 15, the medical device 100 is introduced into the living body through the port 40. In this instance, an arbitrary portion of the medical device 100 is gripped by the gripping section 114 of the first introduction member 110. Furthermore, an arbitrary portion other than the portion gripped by the first introduction member 110, for example, the portion where the extension section 27 of the holding member 20 is provided, is supported by distal portion of the second introduction member 120. The extension section 27 of the holding member 20 is inserted and passed in the insertion lumen 122 of the second introduction member 120. Then, in the condition where the medical device 100, the first introduction member 110 and the second introduction member 120 are assembled, the introduction members 110 and 120 are operated in the manner of pushing them into the living body, thereby introducing the medical device 100 into the living body through the port 40.

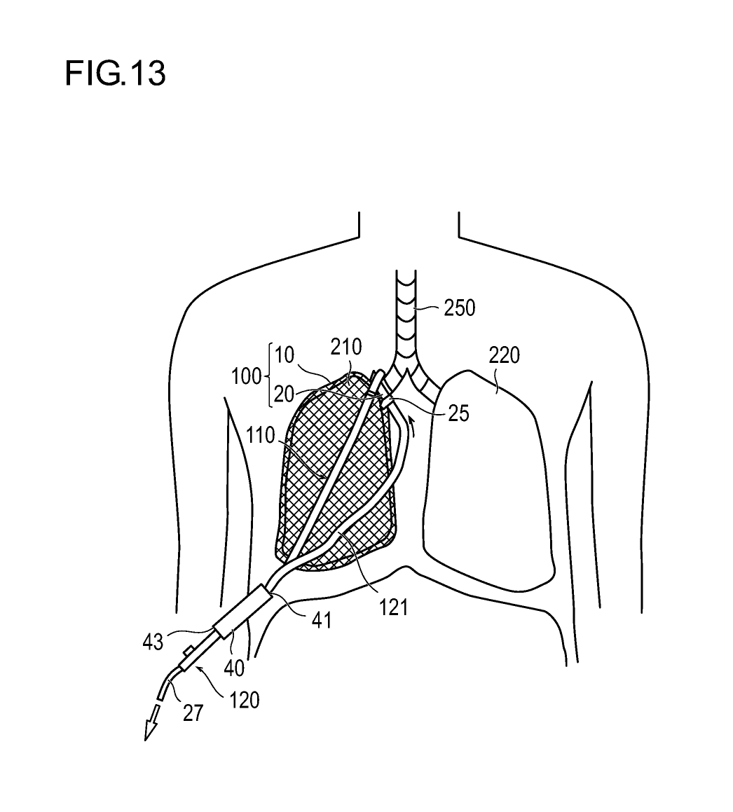

The length of the extension section 27 of the holding member 20 is preliminarily controlled to a predetermined length, prior to the introduction of the medical device 100 into the living body. For example, as will be described later, in order that the length of the extension section 27 can be easily adjusted after the introduction of the medical device 100 into the living body, the length of the extension section 27 can be set to such a length that the extension section 27 is led out through the proximal opening 43 of the port 40 in the condition where the medical device 100 is mounted onto the right lung 210 (see FIG. 13). Incidentally, at the time of performing a procedure of mounting the medical device 100, a proximal portion of the second introduction member 120 is led out to the exterior of the living body through the proximal opening 43 of the port 40, together with the extension section 27 of the holding member 20.

The operation of introducing the medical device 100 into the living body may be carried out as follows. For example, a method may be adopted in which a known thoracoscope for use in the medical field is introduced into the living body through the port 40, and the medical device 100 is advanced while observing an image obtained by the thoracoscope. Or, in the case where the medical device 100, the first introduction member 110 and the second introduction member 120 are provided with radiopacity, the medical device 100 may be advanced radioscopically. Besides, at the time of inserting and passing the medical device 100 in the port 40, the operation may be carried out while deforming the aperture shape of the opening 13 of the medical device 100 by the holding member 20.

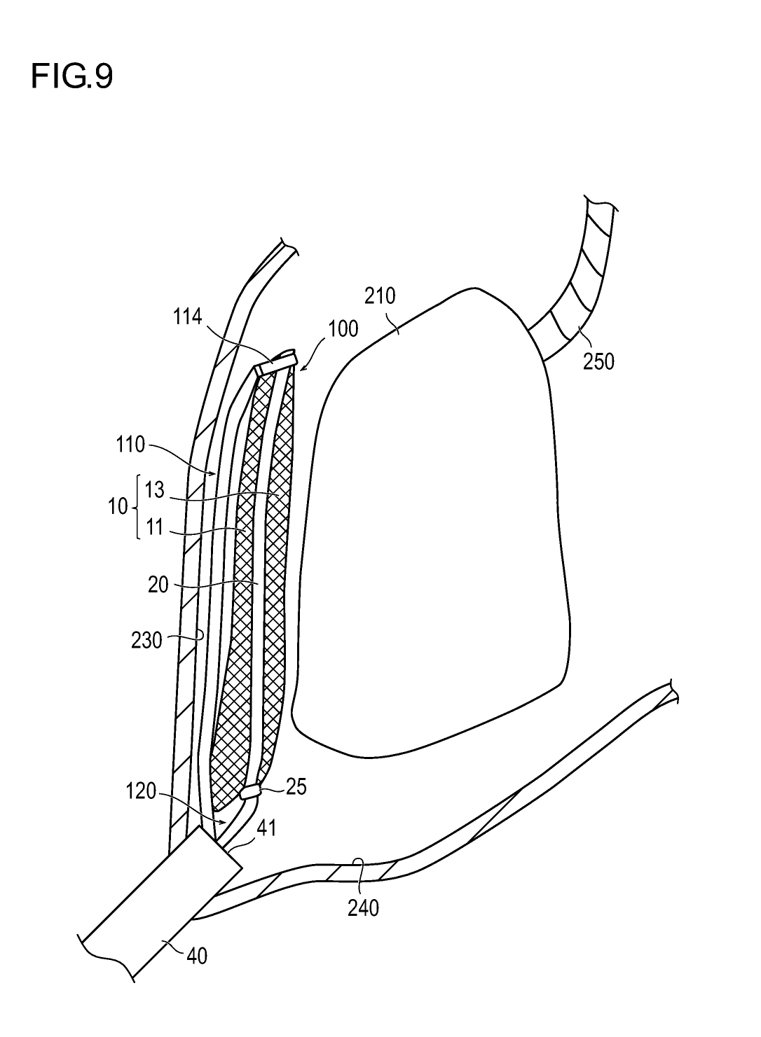

Next, as shown in FIG. 9, after the introduction of the medical device 100 into the inside of the thoracic cage through the distal opening 41 of the port 40, an operation of putting the cover member 10 onto the right lung 210 is performed. For instance, that portion of the holding member 20 which is gripped by the gripping section 124 of the first introduction member 110 is located on the upper lobe side of the right lung 210, whereas that portion of the holding member 20 which is supported by a distal portion of the second introduction member 120 is located on the lower lobe side of the right lung 210. In this instance, in the case where the opening 13 of the cover member 10 is formed in a roughly elliptic shape, the major axis direction of the opening 13 is conformed to the height direction of the right lung 210, whereby the operation of putting the cover member 10 onto the right lung 210 can be carried out more smoothly.

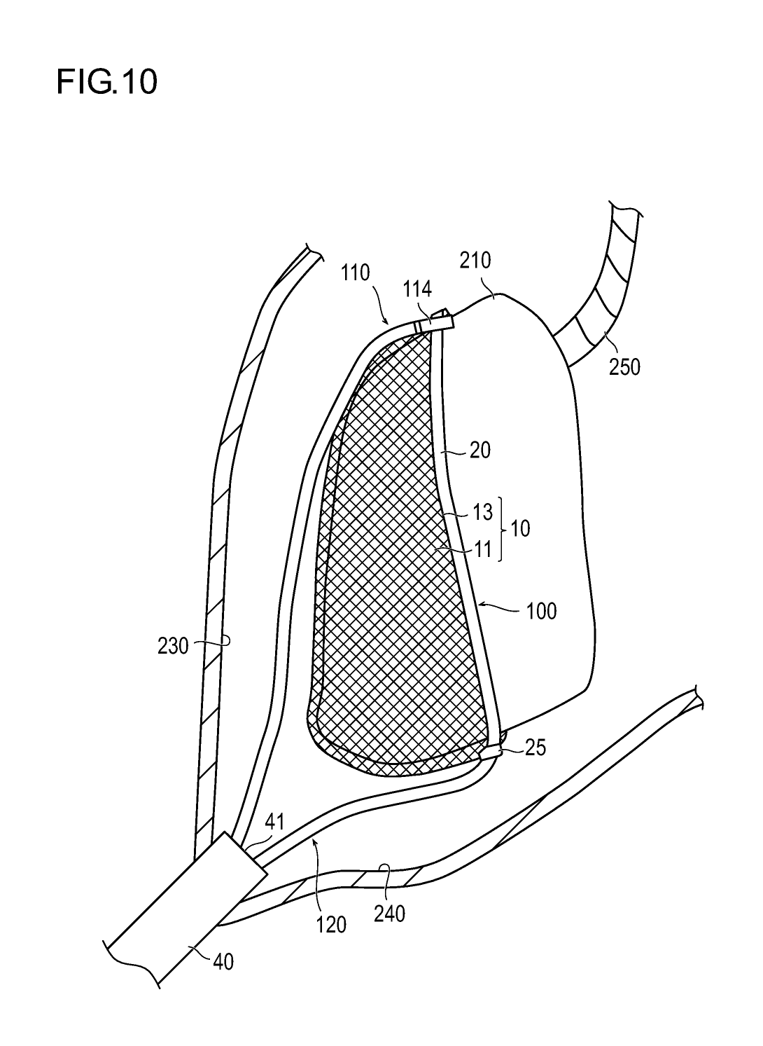

As shown in FIG. 10, a distal-side portion of the first introduction member 110 is advanced between a parietal pleura 230 located at the inner surface of the thoracic cage and a visceral pleura located at the surface of the right lung 210, toward the upper lobe side of the right lung 210. On the other hand, a distal-side portion of the second introduction member 120 is advanced between a diaphragm 240 and the visceral pleura located at the surface of the right lung 210, toward the lower lobe side of the right lung 210. In this instance, since the aperture shape of the opening 13 of the cover member 10 is maintained by the holding member 20, the opening 13 is prevented from being inadvertently deformed to be caught on the right lung 210 or a biorgan present in the surroundings of the right lung 210.

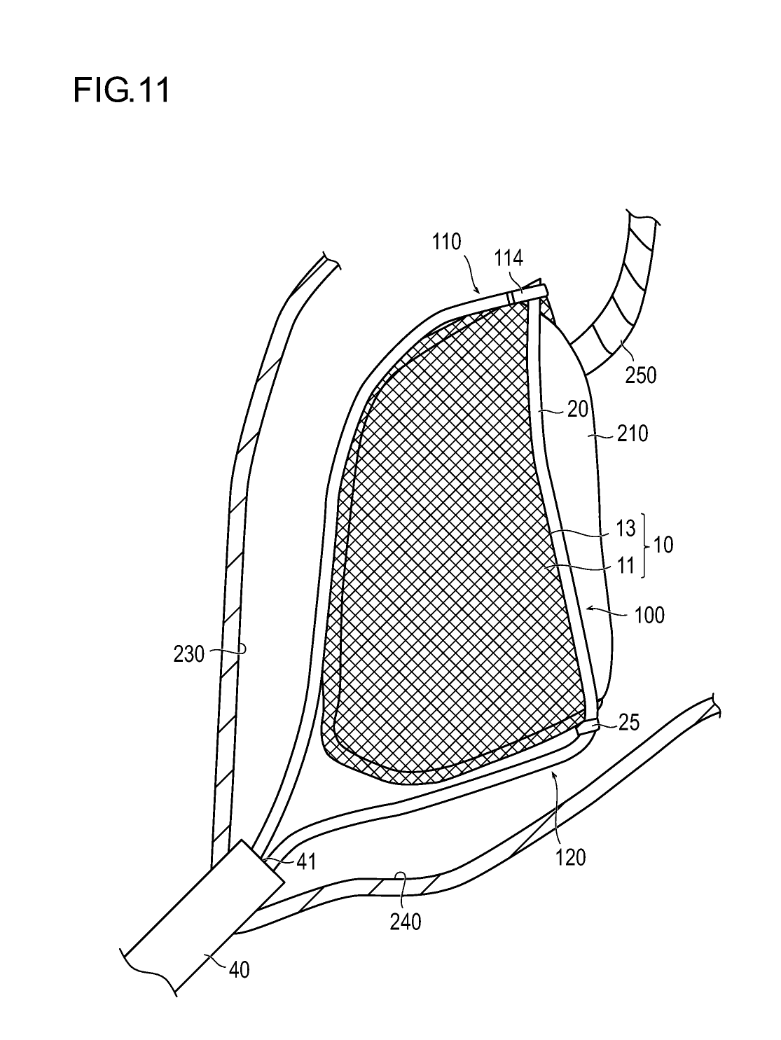

Subsequently, as shown in FIG. 11, the first introduction member 110 and the second introduction member 120 are moved, to proceed with the operation of putting the cover member 10 onto the right lung 210.

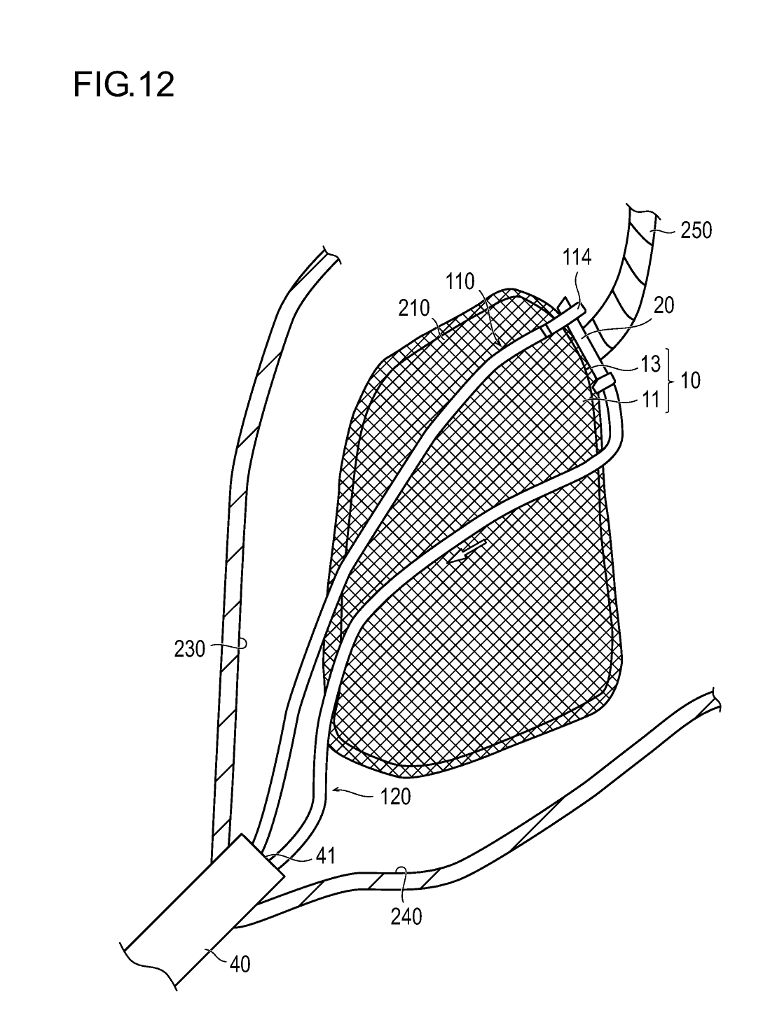

When the right lung 210 has been covered with the cover member 10 and the opening 13 has reached a primary bronchus 250, as shown in FIG. 12, the extension section 27 of the holding member 20 is extended, to reduce the aperture area of the opening 13.

As shown in FIG. 13, the operation of extending the extension section 27 is conducted as follows. In the condition where the holding member 20 is gripped by the first introduction member 110, a proximal portion of the extension section 27 led out from the proximal opening 43 of the port 40 is pulled in the exterior of the living body, and the connecting section 25 of the holding member 20 is slid, to extend the extension section 27. In this instance, the extension section 27 is pulled while pushing the elongated member 121 of the second introduction member 120 distally, whereby the connecting section 25 of the holding member 20 can be moved more smoothly.

When the aperture area of the opening 13 is made to be slightly greater than the diameter of the primary bronchus 250, the operation of controlling the aperture area of the opening 13 is completed. The aperture area of the opening 13 of the cover member 10 is maintained at the as-controlled area by the lock member 30 possessed by the holding member 20.

Thereafter, the gripping of the holding member 20 by the first introduction member 110 is released, and the first introduction member 110 is pulled out of the living body. Subsequently, that predetermined distal-side portion of the extension section 27 which is located in the vicinity of the primary bronchus 250 is cut by the cutter 130 possessed by the second introduction member 120. The portion cut in this manner is held in the insertion lumen 122 of the second introduction member 120. Thereafter, the second introduction member 120 is pulled out. The extension section 27 cut off is taken out of the living body in the state of being held in the insertion lumen 122 of the second introduction member 120.

By the above-mentioned procedure, the operation of putting the cover member 10 onto the right lung 210 is completed. The port 40 provided on the living body is appropriately pulled away after the above-mentioned procedure, and the wound hole formed in the living body is closed appropriately. Incidentally, while the procedure for mounting the medical device 100 onto the right lung 210 has been described above, the operation of putting the medical device 100 onto the left lung 220 can also be carried out according to the same procedure.

As shown in FIG. 14, the right lung 210 with the medical device 100 mounted thereon has a reduced lung volume, as compared with the state before the mounting of the medical device 100. In addition, shrinkability is imparted to the right lung 210, owing to the cover member 10 possessed by the medical device 100.

The medical device 100 and the medical device assembly according to this embodiment as above-described have the following operation and effect.

The aperture shape of the opening 13 of the cover member 10 possessed by the medical device 100 can be changed (deformed) by pushing or pulling mutually opposed arbitrary portions of the holding member 20 provided on the opening 13. In addition, by the holding member 20 it is possible to prevent the aperture area of the opening 13 from being narrowed or enlarged to an unintended size. This ensures that at the time of putting the cover member 10 onto the lung, the opening 13 can be prevented from interfering with and being caught on the lung or a biorgan present in the periphery of the lung. Besides, at the time of introduction into the living body, the aperture shape of the opening 13 can be changed (deformed) comparatively freely, so that the wound hole formed by incision of the living body in preparation for the introduction can be made smaller in size. In addition, by the cover member 10 put on the lung, the lung volume can be reduced, and, further, shrinkability can be imparted to the lung. Therefore, hyperinflation state of the emphysematous lung can be mitigated, and ventilation efficiency of the lung can be enhanced. Consequently, in the treatment of emphysema based on the use of the deformable cover member 10, the operation of putting the cover member 10 onto the lung can be carried out easily and speedily, and a minimally invasive treatment of emphysema can be realized.

Besides, in the case where the holding member 20 includes the main body section 21 disposed along the opening 13, the connecting section 25 provided on the one end side 22 of the main body section 21 and being connectable with the main body section 21, and the extension section 27 extending from the connection position between the main body section 21 and the connecting section 25 toward the other end side 23 of the holding member 20, the following advantage can be attained. The aperture area of the opening 13 can be changed by changing the connection position at which the main body section 21 and the connecting section 25 of the holding member 20 are connected with each other. Therefore, the aperture area of the opening 13 can be easily controlled, even after the introduction of the medical device 100 into the living body. Consequently, the technique based on the use of the medical device 100 can be performed more easily.

The configuration wherein the connecting section 25 of the holding member 20 is movable along the main body section 21 in the state of being connected to the main body section has the following effect. The aperture area of the opening 13 can be controlled by moving the connecting section 25 of the holding member 20 along the main body section 21. This ensures that the operation of controlling the aperture area can be conducted more smoothly.

In the case where the holding member 20 has the lock member 30 which permits the connection position to be changed so as to reduce the aperture area of the opening 13 and prevents the connection position from being so changed as to enlarge the aperture area of the opening 13, the following advantage can be obtained. Owing to the lock member 30, the aperture area of the opening 13 can be prevented from being enlarged inadvertently, after the introduction of the cover member 10 into the living body. Therefore, the cover member 10 can be favorably prevented from falling off the lung. Accordingly, the cover member 10 can be kept indwelling in the living body stably for a long period of time.

Where the extension section 27 of the holding member 20 is separable from the main body section 21, the extension section 27 having become needless after the introduction into the ling body can be separated and taken out of the living body. Accordingly, the medical device 100 can be left indwelling in the living body in a more compact form.

The configuration wherein the cover member 10 is composed of the flexible net is advantageous as follows. By putting the cover member 10 onto the lung, it is possible to reduce the lung volume by the shrinking force of the net, and further to impart shrinkability to the lung. Consequently, a suitable treatment of emphysema can be realized.

Besides, it is possible to provide a medical device 100 suitable for treatment of emphysema, by which the volume of an emphysematous lung can be reduced, and shrinkability can be imparted to the lung.

In the case where the medical device assembly includes the medical device 100, the first introduction member 110 and the second introduction member 120, the following advantage can be attained. In the technique of introducing the medical device 100 into the living body and leaving the medical device 100 indwelling in the living body, the use of the first introduction member 110 and the second introduction member 120 permits the procedure to be carried out more easily, speedily and less invasively.

Where the second introduction member 120 further includes the cutter 130 having the cutting edge part 133 capable of cutting the holding member 20 of the medical device 100, the following advantage can be obtained. Since an arbitrary portion of the holding member 20 can be cut by the cutter 130, it is possible to control the length of the holding member 20, and to cut away an unnecessary portion or portions after the medical device 100 is introduced to the living body. Consequently, convenience of the technique based on the use of the medical device 100 can be enhanced.

Further, in the case where at least one of the holding member 20 of the medical device 100, the first introduction member 110 and the second introduction member 120 is provided with a portion which has radiopacity, the introduction of the component members into the living body can be carried out more smoothly under radioscopic observation.

While the medical device and the medical device assembly according to the present disclosure have been described above by way of embodiments thereof, the present invention is not restricted to the above embodiments, and various alterations are possible on the basis of the claims.

While a mode in which a flexible net is used as the cover member 10 has been described above, any member that is capable of reducing the volume of a lung and imparting shrinkability to the lung can be suitably used as the cover member. For example, deformable mesh materials and those members which are not formed with meshes or the like can also be used as the cover member.

In addition, it suffices for the holding member 20 to have a function of deformably holding the aperture shape of the opening 13 and a function of keeping constant the aperture area before and after the deformation. Thus, the holding member 20 is not restricted only to the member formed from an elastic material or the like, as shown in the embodiment. For instance, a member formed from a plastically deformable material may also be used as the holding member. In this case, the aperture shape of the opening can be reversibly changed (deformed) by plastic deformation.

Besides, as the connecting section of the holding member, those having a connection structure of a recess-projection fitting type can also be adopted. In this case, the connection section can function also as a lock member, and the aperture area of the opening can be reversibly controlled to be larger or smaller. In addition, the holding member may be provided, for example, with a structure such that the extension section is separated from the main body section by pulling the holding member with a predetermined force. Such a configuration ensures that the extension section can be separated without using the second introduction member.

It suffices for the first introduction member to be at least capable of gripping the holding member, and it suffices for the second introduction member to at least permit the extension section of the holding section to be inserted and passed therein. Therefore, the material, layout, shape and the like of the introduction members can be modified, as required. Besides, the cutting function based on the cutter possessed by the second introduction member can be omitted, appropriately. Furthermore, together with the first introduction member and the second introduction member, other introduction member or appliance for assisting the introduction of the medical device may be incorporated in the medical device assembly.

What is claimed is:

1. A medical device comprising:
a cover member including an accommodating section provided so as to be able to accommodate either one of a right lung and a left lung of a living body, the accommodating section being freely deformable according to inflation and deflation of a lung which is accommodated in the state of accommodating the lung therein, and an opening through which the lung is introduced into the accommodating section; and
a holding member which is provided at the opening of the cover member, which deforms aperture shape of the opening by having its oppositely located arbitrary portions brought closer to or away from each other, and by which aperture area of the opening can be held constant before and after the deformation.

2. The medical device according to claim 1,
wherein the holding member includes a main body section disposed along the opening, a connecting section provided on one end side of the main body section and being connectable with the main body section, and an extension section extending from a connection position between the main body section and the connecting section toward the other end side of the holding member; and
the aperture area of the opening can be changed by changing the connection position.

3. The medical device according to claim 2,
wherein the connecting section is so provided as to be movable along the main body section in the state of being connected to the main body section, and
the aperture area of the opening can be changed by moving the connecting section.

4. The medical device according to claim 2, wherein the holding member has a lock member which permits the connection position to be so changed as to reduce the aperture area of the opening and prevents the connection position from being so changed as to enlarge the aperture area of the opening.

5. The medical device according to claim 2, wherein the extension section is so configured as to be separable from the main body section.

6. The medical device according to claim 1, wherein the cover member includes a net which is flexible.

7. The medical device according to claim 1, wherein the medical device is a medical device for treatment of emphysema, the medical device adapted to reduce the volume of an emphysematous lung and to impart shrinkability to the lung.

8. A medical device assembly comprising:
a medical device including
a cover member including an accommodating section provided so as to be able to accommodate either one of a right lung and a left lung of a living body, the accommodating section being freely deformable according to inflation and deflation of a lung which is accommodated in the state of accommodating the lung therein, and an opening through which the lung is introduced into the accommodating section, and
a holding member which is provided at the opening of the cover member, which deforms aperture shape of the opening by having its oppositely located arbitrary portions brought closer to or away from each other, and by which aperture area of the opening can be held constant before and after the deformation;
a first introduction member including a flexible elongated member, a gripping section provided on a distal side of the elongated member and capable of gripping the holding member of the medical device, and a hand operation section provided on a proximal side of the elongated member and operable to control a gripping action of the gripping section; and
a second introduction member including a tubular elongated member formed therein with an insertion lumen in which the extension section of the holding member can be inserted and passed.

9. The medical device assembly according to claim 8, wherein the second introduction member further includes a cutter having a cutting edge part capable of cutting the holding member of the medical device.

10. The medical device assembly according to claim 8, wherein at least one of the holding member of the medical device, the first introduction member, and the second introduction member is provided with a radiopaque part.

11. A method of treating emphysema to reduce a lung volume and to impart shrinkability to a lung by using a cover member which includes an accommodating section being freely deformable according to inflation and deflation of the lung in the state of accommodating the lung therein, wherein the lung is introduced into the accommodating section through an opening which the cover member includes with the aperture area of the opening being held constant and the cover member is kept indwelling in the living body with the lung being covered by the accommodating section.
